(19) United States
(12) Patent Application Publication
(10) Pub. No.: US 2013/0273182 A1
(43) Pub. Date: Oct. 17, 2013

(54) A Herbal Extract for Treating Fatty Liver Disease and a Medication Thereof

(71) Applicants: Po-Lun Wang, Taipei City (TW); Fu-Ming Wang, Taipei City (TW); Shorong-Shii Liou, Kaohsiung City (TW); I-Min Liu, Kaohsiung City (TW); Chia-Ju Chang, Kaohsiung City (TW)

(72) Inventors: Po-Lun Wang, Taipei City (TW); Fu-Ming Wang, Taipei City (TW); Shorong-Shii Liou, Kaohsiung City (TW); I-Min Liu, Kaohsiung City (TW); Chia-Ju Chang, Kaohsiung City (TW)

(21) Appl. No.: 13/445,000

(22) Filed: Apr. 12, 2012

(51) Int. Cl. A61K 36/254; A61P 3/06; A61P 1/16

(52) U.S. Cl. 424/728

(57) ABSTRACT

In the present invention, a herbal extract for treating fatty liver disease and a medication thereof is disclosed, wherein the herbal extract is obtained form Andrographis paniculata and Acanthopanax senticosus.

### BACKGROUND OF THE INVENTION

1. Field of the Invention

The present invention relates to a herbal extract and, more particularly, to a herbal extract for treating fatty liver disease and a medication thereof.

2. Description of the Related Art

People nowadays prefer to eat processed foods or refined foods, which contains high-fat, high-salt or high-sugar. Therefore, redundant fats accumulate in their bodies. In addition the lack of exercises decreases those people's metabolic reaction. Once the physiological metabolism reaction reduces, the incidences to fatty liver will significantly increase. According to statistics, 30% of people in their thirties suffer from fatty liver.

In normal liver, there are 2 to 5 grams of lipids, comprising 50% phospholipids, 20% free fatty acids, 7% cholesterols, and the rest of cholesterol esters, in per 100 grams liver tissue. Fatty liver, also known as fatty liver disease (FLD), is characterized by large vacuoles of lipids, such as triglyceride, accumulating in liver cells, especially when the amount of the triglyceride is more than 5% of total liver weight, or one third area of per unit surface of a hepatic histological section shows steatosis (also called fatty degeneration).

Due to non-obvious symptoms of fatty liver disease, for example non-specific gastrointestinal disorders including upper abdomen pain, loss of appetite, tiredness, flatulency (especially upper abdomen), or the tenderness of the liver area, people aware of their course of FLD only when acute hepatomegaly and fulminant hepatitis occur or take abdominal ultrasonic examinations.

Conventional method for treating FLD is passive and non-drug-depended. First of all, it is necessary to figure out the cause of FLD, such as alcohol abuse, obesity, diabetes, or taking steroid medicine for a long time, and then remove the causes of FLD, by going with diet control and enhance their exercise strength. Finally, depending on their self-curing ability to remove the fat in livers or inhibit the lipids accumulating in livers, especially the triglycerides.

However, the said conventional method for treating FLD may be easy to progress patient's conditions, such as hypercholesterolemia, poor blood circulation, slow bloodstream, and increases incidences to cardiovascular diseases and serious complication, such as chronic hepatitis, fulminant hepatitis, cirrhosis or liver cancer, leading to irreversible consequences.

Furthermore, for people who have hypercholesterolemia and fatty liver at the same time, conventional hypolipidemic agents only can reduce lipids in blood, but fail in reducing lipids in liver cells. Also, the conventional hypolipidemic agents will increase hepatic toxicity after long-term of treatment, so the conventional hypolipidemic agents are unsuitable for people to take for a long period. Besides, for people who only have FLD, the conventional hypolipidemic agents are useless in treatment.

As a result, due to the said inconvenience of the conventional method for treating FLD, it is needed to provide a preferable medication for treating fatty liver disease.

### SUMMARY OF THE INVENTION

The primary objective of this invention is to provide a herbal extract for treating fatty liver disease, which can reduce accumulation of lipids in liver, further reduce the amount of lipid in liver, so as to prevent from fatty liver.

The secondary objective of this invention is to provide a medication for treating fatty liver disease, comprising the said herbal extract obtained from natural plants, and which is capable of inhibiting the formation of fatty liver.

A herbal extract for treating fatty liver disease is obtained by a process comprising steps of drying, by providing an Andrographis paniculata sample and an Acanthopanax senticosus sample, followed by drying the Andrographis paniculata sample and the Acanthopanax senticosus sample till water contents of the Andrographis paniculata sample and Acanthopanax senticosus sample are lower than 10%, to obtain a dry Andrographis paniculata sample and a dry Acanthopanax senticosus sample; extracting, by preparing a mixture including the dry Andrographis paniculata sample and the dry Acanthopanax senticosus sample, and extracting the mixture with ethanol as a solvent under 45 to 55° C. to obtain a liquid extract; and condensation, by condensing the liquid extract to obtain a herbal extract being capable of treating the fatty liver disease.

A medication for treating fatty liver disease comprises the herbal extract as defined in claim 1; and a medical acceptable carrier or excipient.

Further scope of the applicability of the present invention will become apparent from the detailed description given hereinafter. However, it should be understood that the detailed description and specific examples, while indicating preferable embodiments of the invention, are given by way of illustration only, since various others will become apparent from this detailed description to those skilled in the art.

### BRIEF DESCRIPTION OF THE DRAWINGS

The present invention will become more fully understood from the detailed description given herein below and the accompanying drawings which are given by way of illustration only, and thus are not limitative of the present invention, and wherein:

FIG. 1 is a diagram illustrating a manufacture method of a herbal extract in the present invention;

FIG. 2 shows histosection data of liver tissue in HL hamsters of groups (B1) to (B7);

FIG. 3 shows histosection data of kidney tissue in HL hamsters of groups (B1) to (B7).

All figures are drawn for ease of explaining the basic teachings of the present invention only; the extensions of the figures with respect to number, position, relationship, and dimensions of the parts to form the preferred embodiment will be explained or will be within the skill of the art after the following teachings of the present invention have been read and understood. Further, the exact dimensions and dimensional proportions conforming to specific force, weight, strength, and similar requirements will likewise be within the skill of the art after the following teachings of the present invention have been read and understood.

### DETAILED DESCRIPTION OF THE INVENTION

The present invention relates to a herbal extract comprising active substances which can remove lipids that already accumulated in liver and further prevent extra lipid accumulating in liver. The present invention achieves the purpose of treating fatty liver disease. The herbal extract of the present invention includes extracts of Andrographis paniculata and Acanthopanax senticosus. Besides, the extract of Acanthopanax senticosus can ameliorate the decline of kidney index which is caused by the extract of Andrographis paniculata. Also, the extracts of Andrographis paniculata and Acanthopanax senticosus are used to manufacture a medication in the present invention. The medication is not only capable of improving fatty liver, but also to prevent injury of the kidney.

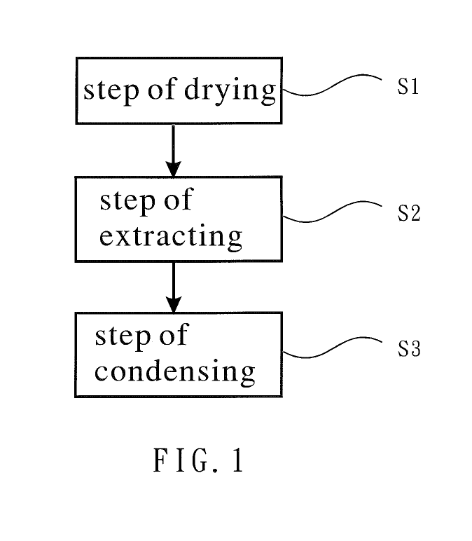

With reference to FIG. 1, the said herbal extract is manufactured by following method, which comprises a step of “drying S1,” a step of “extracting S2,” and a step of “condensing S3”.

In the step of “drying S1,” an Andrographis paniculata sample and an Acanthopanax senticosus sample are prepared and dried individually till the water contents of the Andrographis paniculata sample and the Acanthopanax senticosus sample decrease lower than 10%, in order to obtain a dry Andrographis paniculata sample and a dry Acanthopanax senticosus sample respectively. The Andrographis paniculata sample comprises aerial part of Andrographis paniculata, including stem, leave, and flower parts. The Acanthopanax senticosus sample comprises roots and stems of Acanthopanax senticosus. In an example of the present invention, the Andrographis paniculata sample and the Acanthopanax senticosus sample are carefully washed and dried respectively, and the dry Andrographis paniculata sample and the dry Acanthopanax senticosus sample are obtained. More precisely, drying the Andrographis paniculata sample and the Acanthopanax senticosus sample can be carried by lyophilization, spray drying, evaporation, heating drying or drying at room temperature. In an example of the present invention, the Andrographis paniculata sample and the Acanthopanax senticosus sample are dried at room temperature in the step of “drying S1”, so as to reduce cost and to avoid the deteriorating of active substances of the Andrographis paniculata sample and the Acanthopanax senticosus sample. With such performance, the step of “drying S1” can condense the active substances in the Andrographis paniculata sample and the Acanthopanax senticosus sample, so as to increase the extracting efficiency in the following step of “extracting S2.”

In the step of “extracting S2,” a mixture including the dry Andrographis paniculata sample and the dry Acanthopanax senticosus sample is provided, and extracted with ethanol as a solvent under 45 to 55° C. to obtain a liquid extract. More precisely, the ratio of the dry Andrographis paniculata sample to the dry Acanthopanax senticosus sample in the mixture is anywhere from 1:1 to 1:3, wherein 1 to 4 kilogram of the mixture is extracted by 1 liter of ethanol. In the present embodiment, a mixture is extracted by 95% ethanol at 50° C. under a process of sonication for 8 hours, and preferably repeatedly the extracting three times. After extracting, remove residues of Andrographis paniculata and Acanthopanax senticosus in the solvent, and obtain the liquid extract. With such performance, active substances in the Andrographis paniculata sample and the Acanthopanax senticosus sample are obtained from the liquid extract sufficiently, and therefore the liquid extract of the present invention is potential to be put in use in pharmaceutical industry, by manufacturing the liquid extract into any medicament or health products for inhibiting fatty liver disease.

In the step of “condensing S3,” the liquid extract is condensed to obtain a herbal extract for treating fatty liver disease. As an example, the condensation of the liquid extract can be performed by evaporation or heating drying. In the present embodiment, the liquid extract is condensed via a process of evaporation, in order to remove solvent from the liquid extract and to obtain the herbal extract of the present invention. With such condensation, high concentration of active substances of Andrographis paniculata and Acanthopanax senticosus is successfully obtained in the herbal extract, and the herbal extract is significant in use of pharmaceutical industries.

In the next paragraphs, the benefits of the herbal extract in reducing accumulation of lipids in liver are demonstrated by providing various extracts comprising different compositions, and analyzing the efficiency in DPPH free radical clearance (trial A) and fats eliminating condition (trial B) of those various extracts respectively in the present invention.

Trial (A): DPPH Free Radical Elimination Test

Referring to TABLE 1, there are six groups (A0) to (A5) to test, wherein the group (A0) comprising 10 mg/ml of ascorbic acid, and other five groups comprising extract of a dry Andrographis paniculata sample, a dry Acanthopanax senticosus sample or a combination of dry Andrographis paniculata sample and dry Acanthopanax senticosus sample in various weight ratios respectively. The group (A1) is a liquid extract obtained by extracting 4 kg of dry Andrographis paniculata sample with 1 liter of 95% ethanol. The group (A2) is a liquid extract obtained by extracting 4 kg of dry Acanthopanax senticosus sample with 1 liter of 95% ethanol. The group (A3) is a liquid extract obtained by extracting 4 kg of dry Andrographis paniculata sample and dry Acanthopanax senticosus sample in a weight ratio of 1:1 with 1 liter of 95% ethanol. The group (A4) is a liquid extract obtained by extracting 4 kg of dry Andrographis paniculata and dry Acanthopanax senticosus in a weight ratio of 1:2 with 1 liter of 95% ethanol. The group (A5) is a liquid extract obtained by extracting 4 kg of dry Andrographis paniculata and dry Acanthopanax senticosus in a weight ratio of 1:3 with 1 liter of 95% ethanol.

The liquid extracts of groups (A1) to (A5) is condensed and formulated with physiological saline, and 6 samples are obtained in a concentration in 10 mg/ml. The 6 samples are analyzed in the DPPH free radical elimination ability. 1 ml of the 6 samples and 0.1 mM of α,α-diphenyl-b-picrylhydrazyl (DPPH) are mixed and incubated at room temperature for 30 minutes, to measure the optical density value (OD) of each group under 517 nm. DPPH elimination rates of each sample are further calculated by using Formula I.

$$DPPH Elimination Rate = ( 1 - \frac{O {D}_{517 nm} of sample}{O {D}_{517 nm} of control} ) \times 100 \% \qquad ( Formula I )$$

According to TABLE 1, the DPPH elimination rate of (A5) is higher than that of the others. Hence, the extract of (A5) is selected and used in the Trial B.

TABLE 1
Content in Groups (A0) to (A5)

| | | Percentage of DPPH |
|---|---|---|
| Groups | Content | elimination (%) |
| (A0) | 10 mg/ml ascorbic acid | 96.4 ± 2.7 |
| (A1) | 10 mg/ml of extract of only dry | 42.1 ± 1.8 |
| | Andrographis paniculata | |
| (A2) | 10 mg/ml of extract of only dry | 57.3 ± 1.7 |
| | Acanthopanax senticosus | |
| (A3) | 10 mg/ml of herbal extract* | 68.7 ± 2.1 |
| (A4) | 10 mg/ml of herbal extract* | 76.4 ± 1.9 |
| (A5) | 10 mg/ml of herbal extract* | 87.3 ± 2.6 |

*A weight ratio between the dry Andrographis paniculata sample and the dry Acanthopanax senticosus sample of the group (A3) is 1:1, the weight ratio of the group (A4) is 1:2, and the weight ratio of the group (A5) is 1:3.

Trial (B): Fats Eliminating Test

In the present embodiment, male, around 100 to 150 grams and 4-weeks-old Golden Syrian hamsters purchased from National Laboratory Animal Center in Taiwan are prepared, housed at a standard laboratory environment, such as keeping 25±1° C. and with a 12 hours light/dark cycle, and fed with normal diet till they are 6-weeks-old, and then fed high-cholesterol diet (TestDiet® Formula 5TJT, containing 1% cholesterol) instead of the normal diet for next 4 weeks. With such arrangement, hyperlipidemic hamsters (HL hamsters), whose triglyceride index (TG) in blood is higher than 200 mg/dl, and total cholesterol (TC) index is higher than 260 mg/dl, are obtained.

In addition, 4-weeks-old normal Golden Syrian hamsters are also prepared, which are fed with normal diet till they are 10-week-old. The normal diet comprises 18% of raw protein, 10% of water, 6% of raw fats, 6% of ash content, 6% of raw fiber, and 54% of soluble nitrogen free extract.

With reference to TABLE 2, normal hamsters set into group (B1) as a negative control group, and HL hamsters are arranged into 6 groups, wherein group (B2) is positive control group, and groups (B3) to (B7) are experimental groups. Each group includes eight hamsters. In the embodiment of the present invention, each group is fed with the feeding contents according to TABLE 2 via stomach tube, and recording the weight of each group at 4^{th} week and 8^{th} week. The volume of the feeding content is 2 ml per kilogram weight of hamster in each group. The group (B1) is fed with physiological saline; the group (B2) is fed with physiological saline; the groups (B3) to (B5) are fed with different concentration (100, 300 and 500 mg/per kilogram of weight/day) of herbal extract in a weight ratio of 1:3 between the dry Andrographis paniculata sample and the dry Acanthopanax senticosus sample, respectively; the group (B6) is fed with 500 mg/per kilogram of weight/day of extract of Andrographis paniculata; and the group (B7) is fed with 100 mg/per kilogram of weight/day of a commercial drug, LipoCol Forte®.

There have no significant difference between the groups (B3) to (B6) and the group (B7) in the average weight of hamsters. It presents that the extract of Andrographis paniculata or the extract including Andrographis paniculata and Acanthopanax senticosus, are capable of reducing the weight of individual. The weights of the hamsters are measured after eight weeks, the hamsters of each group is dissected to collect liver and kidney tissue. In the present embodiment, histosection data of the liver tissue and the kidney tissue of hamsters are prepared and analyzed to determine the lipid accumulation in liver and the kidney injury in each group.

TABLE 2
Groups Assignment in Trial (B)

| | | weight (gram) | | |
|---|---|---|---|---|
| Groups | feeding content | 0 week | 4^{th} week | 8^{th} week |
| (B1) | physiological saline | 73.22 ± 5.91* | 75.72 ± 8.93* | 76.33 ± 3.92* |
| (B2) | physiological saline | 109.22 ± 9.26 | 117.62 ± 8.85 | 121.38 ± 8.39 |
| (B3) | 100 mg/kg/day of | 108.84 ± 9.92 | 101.72 ± 6.43* | 98.84 ± 5.16* |
| | herbal extract | | | |
| (B4) | 300 mg/kg/day of | 102.32 ± 8.88 | 96.53 ± 5.84* | 92.76 ± 7.01* |
| | herbal extract | | | |
| (B5) | 500 mg/kg/day of | 103.79 ± 6.01 | 94.72 ± 6.71* | 90.21 ± 9.28* |
| | herbal extract | | | |
| (B6) | 500 mg/kg/day of | 104.46 ± 5.76 | 95.41 ± 5.92* | 92.34 ± 8.62* |
| | extract of Andrographis | | | |
| | paniculata | | | |
| (B7) | 100 mg/kg/day of | 105.43 ± 7.72 | 92.91 ± 5.31* | 90.19 ± 6.98* |
| | LipoCol Forte ® | | | |

*p < 0.05, presents there have significant difference when compared to group (B2)

With reference to TABLEs 3 and 4, the plasma levels of the groups (B1) to (B7) in 4^{th} week and 8^{th} week are shown. The plasma levels include the levels of glucose, glutamic oxaloacetate transaminase (GOT), glutamic pyruvic trnsminase (GPT), blood urea nitrogen (BUN), and creatinine.

TABLE 3
Plasma Level of Group (B1) to (B7) in 4^{th} Week

| | GOT | GPT | BUN | Creatinine | Glucose |
|---|---|---|---|---|---|
| Groups | (U/L) | (U/L) | (mg/dl) | (mg/dl) | (mg/dl) |
| (B1) | 50.21 ± 15.30** | 78.00 ± 4.21** | 9.72 ± 2.35* | 0.07 ± 0.06* | 84.28 ± 13.16* |
| (B2) | 372.31 ± 11.27 | 1125.64 ± 13.43 | 16.67 ± 5.92 | 0.16 ± 0.05 | 129.54 ± 12.15 |
| (B3) | 278.23 ± 10.21* | 713.26 ± 15.93* | 13.11 ± 2.79 | 0.10 ± 0.02 | 95.39 ± 10.17* |
| (B4) | 263.19 ± 9.84* | 672.21 ± 11.07* | 12.53 ± 2.37 | 0.08 ± 0.03 | 91.23 ± 10.52* |
| (B5) | 256.62 ± 9.26* | 631.17 ± 12.73* | 12.12 ± 2.12 | 0.06 ± 0.02* | 92.76 ± 10.43* |
| (B6) | 26861 ± 7.57* | 669.29 ± 11.44* | 13.27 ± 2.46 | 0.07 ± 0.02* | 93.46 ± 11.29* |
| (B7) | 253.12 ± 9.18* | 628.21 ± 10.02* | 10.13 ± 3.84 | 0.06 ± 0.02* | 96.00 ± 13.21* |

*p < 0.05,
**p < 0.01, presents there have significant difference when compared to group (B2)

In TABLE 3, the plasma levels of the groups (B1) to (B7) in 4^{th} week are slightly decreased, and a significant difference between the group (B2) and the groups (B3) to (B5).

TABLE 4
Plasma Level of Group (B1) to (B7) in 8^{th} Week

| | GOT | GPT | BUN | Creatinine | Glucose |
|---|---|---|---|---|---|
| Groups | (U/L) | (U/L) | (mg/dl) | (mg/dl) | (mg/dl) |
| (B1) | 57.36 ± 14.75** | 82.27 ± 4.65** | 9.76 ± 2.14** | 0.06 ± 0.04* | 85.32 ± 12.61* |
| (B2) | 396.42 ± 11.93 | 1269.64 ± 14.65 | 17.21 ± 4.76 | 0.19 ± 0.09 | 128.39 ± 13.62 |
| (B3) | 276.21 ± 9.74* | 702.19 ± 10.25* | 12.97 ± 2.81* | 0.08 ± 0.04* | 96.24 ± 11.12* |
| (B4) | 260.15 ± 9.32* | 665.83 ± 9.81* | 12.19 ± 2.10* | 0.08 ± 0.06* | 92.27 ± 10.39* |
| (B5) | 250.36 ± 9.12* | 629.28 ± 9.31* | 11.19 ± 2.43* | 0.07 ± 0.03* | 92.32 ± 10.28* |
| (B6) | 256.43 ± 9.46* | 652.32 ± 9.67* | 12.11 ± 2.37* | 0.08 ± 0.04* | 94.36 ± 11.45* |
| (B7) | 233.10 ± 9.17** | 601.22 ± 9.05* | 10.07 ± 2.76* | 0.06 ± 0.03* | 90.19 ± 10.14* |

*p < 0.05,
**p < 0.01, presents there have significant difference when compared to group (B2)

Referring to TABLE 4, the liver index of the group (B5) and (B7) in 8^{th} week are lower than the group (B2). Comparing to the group (B2), the GOT of the group (B5) is 37.84±2.38% decreased, the GPT of the group (B5) is 50.43±5.28% decreased; the GOT of the group (B7) is 58.80±4.26% decreased, the GPT of the group (B7) is 50.65±4.96% decreased. Besides, in comparison with the kidney index of the groups (B3) to (B6), the group (B6) is higher than others, it is indicated that the extract of Andrographis paniculata causes the injury of the HL hamsters. Comparing with the group (B6), the kidney index of the group (B5) only declines slightly, it is indicated the herbal extract of the present invention can not only achieve the liver index of the group (B7), but also improve the kidney injury caused by the extract of Andrographis paniculata.

The kidney index of the group (B5) and (B7) in 8^{th} week are lower than the group (B2). Comparing to the group (B2), the BUN of the group (B5) is 34.98±2.71% decreased, the creatinine of the group (B5) is 12.50±2.14% decreased; the BUN of the group (B7) is 41.48±3.26% decreased, the creatinine of the group (B7) is 25.00±2.21% decreased. In addition, compared with the group (B2), the decreasing percentage of glucose level in the group (B5) is 28.09±2.09%, and the group (B7) is 28.05±3.12%.

With reference to TABLEs 5 and 6, the levels of blood lipids of the groups (B1) to (B7) in 4^{th} week and 8^{th} week are shown. The levels of blood lipids include triglyceride (TG), total cholesterol (TC), high-density lipoprotein cholesterol (HDL), low-density lipoprotein cholesterol (LDL), and ratio of LDL and HDL (LDL/HDL).

TABLE 5
Levels of Blood Lipid of Group (B1) to (B7) in 4^{th} Week

| | TG | TC | HDL | LDL | |
|---|---|---|---|---|---|
| Groups | (mg/dl) | (mg/dl) | (mg/dl) | (mg/dl) | LDL/HDL |
| (B1) | 83.67 ± 9.31** | 126.67 ± 5.24** | 94.57 ± 5.18** | 21.79 ± 1.17** | 0.23 ± 0.17** |
| (B2) | 536.52 ± 15.21 | 926.51 ± 15.64 | 42.28 ± 5.62 | 635.37 ± 8.54 | 15.03 ± 0.35 |
| (B3) | 472.64 ± 12.43* | 782.39 ± 16.72* | 54.59 ± 5.94* | 563.21 ± 8.32* | 10.31 ± 0.28* |
| (B4) | 453.83 ± 12.28* | 761.21 ± 14.42* | 55.38 ± 5.63* | 527.36 ± 7.80* | 9.52 ± 0.32* |
| (B5) | 427.26 ± 13.29* | 725.83 ± 14.19* | 55.64 ± 5.32* | 505.43 ± 7.82* | 9.08 ± 0.29* |
| (B6) | 445.31 ± 12.62* | 763.47 ± 13.24* | 55.41 ± 5.27* | 516.28 ± 8.35* | 9.89 ± 0.31* |
| (B7) | 396.23 ± 12.14* | 706.46 ± 13.27* | 56.39 ± 4.29* | 482.21 ± 8.19* | 8.55 ± 0.26* |

*p < 0.05,
**p < 0.01, presents there have significant difference when compared to group (B2)

TABLE 6
Levels of Blood Lipid of Group (B1) to (B7) in 8^{th} Week

| | TG | TC | HDL | LDL | |
|---|---|---|---|---|---|
| Groups | (mg/dl) | (mg/dl) | (mg/dl) | (mg/dl) | LDL/HDL |
| (B1) | 83.97 ± 8.75** | 129.21 ± 5.39** | 92.32 ± 5.26** | 25.29 ± 2.39** | 0.27 ± 0.23** |
| (B2) | 543.39 ± 14.46 | 937.42 ± 13.54 | 40.16 ± 5.32 | 642.35 ± 9.12 | 15.99 ± 0.39 |
| (B3) | 470.62 ± 13.27* | 769.49 ± 13.29* | 56.19 ± 5.29* | 559.42 ± 8.41* | 9.96 ± 0.34* |
| (B4) | 476.53 ± 10.17* | 757.28 ± 13.71* | 58.27 ± 5.25* | 518.26 ± 7.91* | 8.89 ± 0.35* |
| (B5) | 418.73 ± 10.53* | 709.17 ± 13.37* | 59.59 ± 5.43* | 496.72 ± 7.29* | 8.33 ± 0.30* |
| (B6) | 446.56 ± 10.28* | 747.43 ± 13.62* | 57.41 ± 5.37* | 507.46 ± 7.62* | 8.65 ± 0.28* |
| (B7) | 374.59 ± 10.19* | 682.29 ± 12.24* | 60.13 ± 4.86* | 471.29 ± 7.31* | 7.84 ± 0.28* |

*p < 0.05,
**p < 0.01, presents there have significant difference when compared to group (B2)

Referring to TABLE 6, the level of blood lipids of the group (B5) and (B7) in 8^{th} week are lower than the group (B2). Comparing to the group (B2), the TG of the group (B5) is 24.34±2.21% decreased, the TC of the group (B5) is 22.94±2.97% decreased, the LDL of the group (B5) is 22.68±2.76% decreased; the TG of the group (B7) is 27.22±3.14% decreased, the TC of the group (B7) is 31.06±2.83% decreased, the LDL of the group (B7) is 26.63±3.12% decreased. Accordingly, the decreasing efficacy of blood lipid of the group (B5) is similar to the group (B7). It is suggested that the herbal extract, which ratio of the dry Andrographis paniculata sample and the dry Acanthopanax senticosus sample is 1:3 in the present invention, is capable of decreasing the blood lipid.

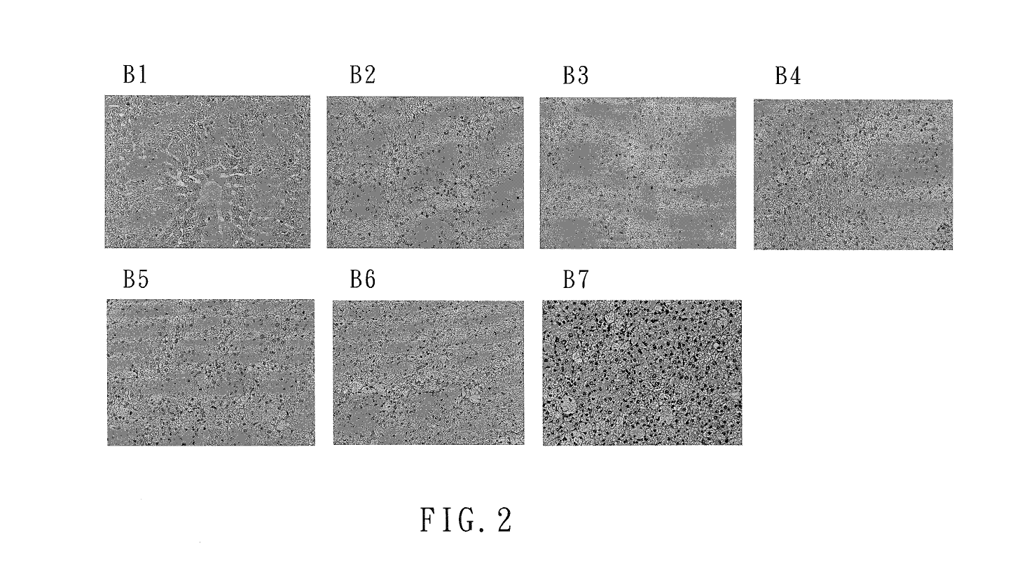

For further proving the therapeutic effects of the herbal extract in inhibiting of FLD, in the embodiment of the present invention, the pathological symptom of the histosection data of the groups (B1) to (B7) are evaluated by pathological doctors who were not aware of the assignment of each group, and the liver pathological symptoms includes hepatocellular hydropic degeneration, central lobular necrosis, hepatic lipidosis, and hepatic fibrosis. Referring to FIG. 2, it shows micrographs of the histosection data of liver tissue in the groups (B1) to (B7), wherein the magnification power is 200×. To reducing the bias of individuals, the liver tissues of each hamster are collected from the approximate part at right lobe of liver tissue, being 1 cm² of area, followed by fixed with 10% neutral formalin solution, embodied in paraffin, and stained by hematoxylin and eosin stain (HE stain).

In the present invention, the scoring of liver pathogenic symptom in each hamster is according to the following standard: “score 0” presents no pathological change is observed, “score 1” presents 0˜25% of pathological change area in the per unit of section is observed, “score 2” presents 25˜50% of pathological change area in the per unit of section is observed, “score 3” presents 50˜75% of pathological change area in the per unit of section is observed, and “score 4” presents 75˜100% of pathological change area in the per unit of section is observed.

It is noted that the hepatocellular hydropic degeneration is characterized by the vacuoles within liver tissues. Accordingly, the larger area of vacuoles in the histosection micrograph is observed, the more serious the hepatocellular hydropic degeneration of the liver tissue it does.

It is noted that the hydropic degeneration is characterized by a large amount of bubble or liquid in cytoplasm caused by abnormal metabolism in liver cells, wherein the symptoms of hydropic degeneration is reversible if causes thereof has removed.

It is noted that the central lobular necrosis is characterized by irreversible necrosis around the central vein leading to condensation of nucleus and cytoplasm.

It is noted that the hepatic fibrosis is characterized by a large amount of cell necrosis occurred at central vein developing into excessive connective tissue in the liver, which is an irreversible symptom.

TABLE 7
Scoring of Liver Pathological Symptoms of Groups (B1) to (B7)

| | hepatocellular | | | |
|---|---|---|---|---|
| | hydropic | central lobular | hepatic | hepatic |
| Groups | degeneration | necrosis | lipidosis | fibrosis |
| (B1) | 0.5 ± 0.1 | 0.5 ± 0.2 | 0.2 ± 0.1 | 0.3 ± 0.2 |
| (B2) | 4.8 ± 0.5 | 4.1 ± 0.6 | 4.8 ± 0.6 | 4.9 ± 0.3 |
| (B3) | 3.8 ± 0.2 | 3.5 ± 0.4 | 3.5 ± 0.3 | 3.7 ± 0.2 |
| (B4) | 3.2 ± 0.3 | 3.4 ± 0.3 | 3.3 ± 0.2 | 3.5 ± 0.3 |
| (B5) | 2.8 ± 0.2 | 3.0 ± 0.2 | 3.0 ± 0.2 | 3.2 ± 0.2 |
| (B6) | 3.3 ± 0.2 | 3.3 ± 0.2 | 3.3 ± 0.2 | 3.4 ± 0.1 |
| (B7) | 2.6 ± 0.2 | 2.9 ± 0.2 | 2.8 ± 0.2 | 3.0 ± 0.2 |

With referring to TABLE 7, a significant difference (p<0.05) between the groups (B3) to (B5) and the group (B2) is observed. It is suggested that the dosage of the herbal extract in present invention is preferably at 100 to 500 mg for per kilogram of body weight per day.

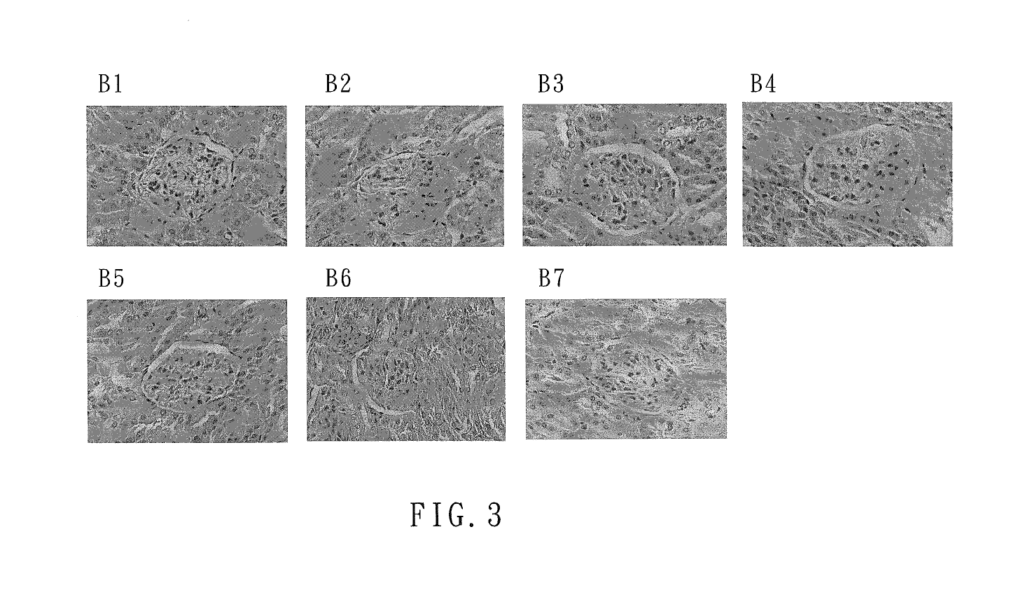

For further proving the inhibition of fatty liver in individual without kidney injury, the kidney injury symptom of histosection data of the groups (B1) to (B7) are evaluated by pathological doctors who were not aware of the assignment of each group, and the kidney injury symptom includes degeneration of renal tubular epithelium, tubular epithelium fatty change, and renal tubular necrosis. Referring to FIG. 3, it shows micrographs of the histosection data of kidney tissue in the groups (B1) to (B7), wherein the magnification power is 200×. To reducing the bias of individuals, the kidney tissues of each hamster are collected from the left side of kidney tissue, being 1 cm² of area, followed by fixed with 10% neutral formalin solution, embodied in paraffin, and stained by hematoxylin and eosin stain (HE stain).

In the present invention, the scoring of kidney injury symptom in each hamster is according to the following standard: “score 0” presents no pathological change is observed, “score 1” presents 0˜25% of pathological change area in the per unit of section is observed, “score 2” present 25˜50% of pathological change area in the per unit of section is observed, “score 3” present 50˜75% of pathological change area in the per unit of section is observed, and “score 4” present 75˜100% of pathological change area in the per unit of section is observed.

TABLE 8
Scoring of Kidney Pathological Symptoms of Groups (B1) to (B7)

| | Degeneration of renal | Tubular epithelium | Renal tubular |
|---|---|---|---|
| Groups | tubular epithelium | fatty change | necrosis |
| (B1) | 0 | 0 | 0 |
| (B2) | 0.3 ± 0.1 | 0.3 ± 0.2 | 0.3 ± 0.1 |
| (B3) | 0.1 ± 0.2 | 0.1 ± 0.1 | 0.1 ± 0.1 |
| (B4) | 0.1 ± 0.1 | 0.1 ± 0.2 | 0.1 ± 0.2 |
| (B5) | 0.1 ± 0.2 | 0.1 ± 0.1 | 0.1 ± 0.1 |
| (B6) | 0.2 ± 0.1 | 0.4 ± 0.2 | 0.2 ± 0.2 |
| (B7) | 0.1 ± 0.1 | 0.1 ± 0.1 | 0.1 ± 0.1 |

Referring to FIG. 3, the groups (B2) and (B6) both show kidney injury symptom, especially the group (B6) shows clearly kidney injury in comparison with the groups (B3) to (B5). It is suggested that the herbal extract of the groups (B3) to (B5) not only prevent the kidney injury caused by the extract of Andrographis paniculata, also reduce the kidney injury caused by FLD. With referring to TABLE 8, there also have a significant difference (p<0.05) between the groups (B3) to (B5) and the group (B2), wherein the group (B5) is more efficient in reducing the kidney injury caused by FLD. It is suggested that the herbal extract in the present invention, not only can inhibit FLD, but also solves the kidney injury problem, which caused by the extract of Andrographis paniculata.

In the present invention, the said herbal extract is capable of reducing the amount of TC, TG, and LDL in liver cell, so as to prevent the occurence of FLD. Besides, the said herbal extract also can ease off the decline of kidney index, and avoid the kidney injury caused by Andrographis paniculata.

It is suggested that the herbal extract of the present invention is potential to be applied to pharmaceutical industry, being an active substance of medication or health products for inhibiting FLD. In the present invention, the herbal extract can be given to any target individually, or combined with any acceptable excipients, for example carriers or other ingredients, and is capable of being further manufactured into any form of medicament, such as pill, capsule, powder, solution and pastil for easy and convenient delivery to targets. The herbal extract also can be made into a suitable form, such as mixing the herbal extract with other foods or drinks, for individual to take orally.

The medication of the present invention comprises the extract of dry Andrographis paniculata and dry Acanthopanax senticosus in a weight ratio of 1:1 to 1:3, particularly extracted by ethanol, and is preferably delivered to a target once a day, with a dosage of 100 to 500 mg per kilogram of body weight and with a period of treatment lasting for 8 weeks. Therefore, the medication is capable of helping physiological activity, so as to improve the liver and kidney index and prevent the kidney injury problem caused by Andrographis paniculata.

In summary, through the present invention, a herbal extract for treating fatty liver disease is provided, by inhibiting the accumulation of lipid in liver cell, and further reducing the lipid amount in liver or preventing the occurence of FLD efficiently. With the herbal extract of the present invention, a medication or health products comprising the herbal extract are also easily obtained, and which has natural medical properties in treating FLD, and will be easy to put to used in pharmaceutical industries.

Thus, since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A herbal extract for treating fatty liver disease, which is obtained by a process comprising steps of:
drying, by providing an Andrographis paniculata sample and an Acanthopanax senticosus sample, followed by drying the Andrographis paniculata sample and the Acanthopanax senticosus sample respectively till water contents of the Andrographis paniculata sample and the Acanthopanax senticosus sample are lower than 10%, to obtain a dry Andrographis paniculata sample and a dry Acanthopanax senticosus sample respectively;
extracting, by preparing a mixture including the dry Andrographis paniculata sample and the dry Acanthopanax senticosus sample, and extracting the mixture with ethanol as a solvent under 45 to 55° C. to obtain a liquid extract; and
condensing the liquid extract to obtain a herbal extract.

2. The herbal extract for treating fatty liver disease as defined in claim 1, wherein a ratio of the dry Andrographis paniculata sample to the dry Acanthopanax senticosus sample in the mixture of the step of extracting is anywhere from 1:1 to 1:3.

3. The herbal extract for treating fatty liver disease as defined in claim 1, wherein 1 to 4 kilogram of the mixture is extracted by 1 liter of ethanol in the step of extracting.

4. The herbal extract for treating fatty liver disease as defined in claim 2, wherein 1 to 4 kilogram of the mixture is extracted by 1 liter of ethanol in the step of extracting.

5. The herbal extract for treating fatty liver disease as defined in claim 1, wherein the step of extracting is processed via sonication.

6. The herbal extract for treating fatty liver disease as defined in claim 1, wherein the temperature of the step of extracting is 50° C.

7. The herbal extract for treating fatty liver disease as defined in claim 1, wherein the extracting time of the step of extracting is 8 hours.

8. A medication for treating fatty liver disease, comprising:
a herbal extract as defined in claim 1; and
a medical acceptable carrier or excipient.

9. The medication for treating fatty liver disease as defined in claim 8, wherein the said herbal extract is obtained by a process comprising steps of:
drying, by providing an Andrographis paniculata sample and an Acanthopanax senticosus sample, followed by drying the Andrographis paniculata sample and the Acanthopanax senticosus sample respectively till water contents of the Andrographis paniculata sample and the Acanthopanax senticosus sample are lower than 10%, to obtain a dry Andrographis paniculata sample and a dry Acanthopanax senticosus sample respectively;
extracting, by preparing a mixture including the dry Andrographis paniculata sample and the dry Acanthopanax senticosus sample, and extracting the mixture with ethanol as a solvent under 45 to 55° C. to obtain a liquid extract; and
condensing the liquid extract to obtain a herbal extract.

10. The medication for treating fatty liver disease as defined in claim 8, wherein a ratio of the dry Andrographis paniculata sample to the dry Acanthopanax senticosus sample in the mixture of the step of extracting is anywhere from 1:1 to 1:3.

11. The medication for treating fatty liver disease as defined in claim 8, wherein the medication is an oral agent.

12. The medication for treating fatty liver disease as defined in claim 8, wherein the medication is in the form of a pill, pastil, powder, capsule or solution.

13. The medication for treating fatty liver disease as defined in claim 8, wherein a dosage of the medication is 100 to 500 mg/per kilogram of body weight per day.
